(19) United States
(12) Patent Application Publication
(10) Pub. No.: US 2015/0364023 A1
(43) Pub. Date: Dec. 17, 2015

(54) MONITORING SYSTEM OF MOTION SENSING CARPETS

(71) Applicants: Yeh-Liang Hsu, Taoyuan City (TW); SEDA CHEMICAL PRODUCTS CO., LTD., New Taipei City (TW)

(72) Inventors: Yeh-Liang HSU, Taoyuan City (TW); Wei-Kuan WANG, New Taipei City (TW); Kai-Wei CHANG, Miaoli County (TW); Yu-Wei LIU, New Taipei City (TW); Wei-Yi CHANG, Taipei City (TW)

(73) Assignees: SEDA CHEMICAL PRODUCTS CO., LTD., New Taipei City (TW); Yeh-Liang Hsu, Taoyuan City (TW)

(21) Appl. No.: 14/681,308

(22) Filed: Apr. 8, 2015

(30) Foreign Application Priority Data
Jun. 16, 2014 (TW) 103120735

(51) Int. Cl. G08B 21/04; A61B 5/11

(52) U.S. Cl. CPC G08B 21/0461; A61B 5/1117

(57) ABSTRACT

The present invention is to provide a monitoring system including a plurality of motion sensing carpets and a monitoring device (e.g., a computer) electrically connected to one of the motion sensing carpets serving as a control unit while all the other motion sensing carpets directly or indirectly joined to the control unit serves as auxiliary units. The monitoring device is able to carry out a topology algorithm and then establish a topology matrix of the motion sensing carpets in a stepwise manner so as to obtain the relative location of each motion sensing carpet. When any of the motion sensing carpets is subjected to pressure caused by a senior member or child in the family toppling over thereon (e.g., an accident) and generates a sensing signal, the monitoring device can rapidly know from the topology matrix the exact location of the accident according to the sensing signal.

### FIELD OF THE INVENTION

The present invention relates to a monitoring system, more particularly to a monitoring system including a plurality of motion sensing carpets and a monitoring device (e.g., a computer) electrically connected to one of the motion sensing carpets serving as a control unit while all the other motion sensing carpets directly or indirectly joined to the control unit serves as auxiliary units. The monitoring device is able to carry out a topology algorithm and then establish a topology matrix of the motion sensing carpets in a stepwise manner so as to obtain the relative location of each motion sensing carpet. When any of the motion sensing carpets is subjected to pressure caused by a senior member or child in the family toppling over thereon and generates a sensing signal, the monitoring device can rapidly know from the topology matrix the exact location of the accident according to the sensing signal.

### BACKGROUND OF THE INVENTION

The population pyramid is changing worldwide as a result of declining birth rate and improvements in the medical environment. The percentage of the elderly population, in particular, has risen significantly. In 1950, a senior citizen was reared by an average of twelve people in the labor force. As the population pyramid changes, however, the ratio of the latter to the former is lowered on a yearly basis such that the burden on the labor force is increasing. In Taiwan, for example, the aforesaid ratio has dropped to 7:1 and is estimated to reach 2.7:1 in twenty years. More attention, therefore, should be paid to the physical and mental health and medical care of the elderly. In fact, how to create an environment where the aged can lead comfortable, cheerful, and carefree lives while those in the labor force are allowed to devote themselves to work without having to worry about the wellbeing of their senior family members is a subject that concerns us all.

Physiological aging takes place as we grow old. An aged person not only may respond more slowly to the outside world, but also may become less capable of performing various body movements. In many cases, physiological aging can cause inconvenience to a person's daily life, especially a sick person's. Such inconveniences may also give rise to danger and hence should be dealt with seriously. An elderly person, when not tended to, may topple over, bump into an object by accident, or even collapse to the ground due to a sudden physiological condition. To prevent the foregoing scenarios, in which the danger may escalate without timely help, more and more importance is attached to domestic safety, telecare, and like issues, and because of that, related applications and technologies are being developed rapidly. A notable example of products developed to cope with the aforesaid scenarios is the motion sensing carpet.

Typically, a conventional motion sensing carpet is provided therein with a sensor module. When subjected to pressure, the sensor module sends a sensing signal to a monitoring device (e.g., a computer) in order for a caregiver (e.g., a family member in the labor force who is in charge of caregiving or a professional in a nursing home) to know via the monitoring device the current activity of the elderly person being monitored and take necessary actions as soon as an abnormal condition is identified. While a conventional motion sensing carpet is indeed helpful in notifying a caregiver of the emergence of an accident, it has limitations in use. When a conventional motion sensing carpet is laid over a small area, a caregiver spotting an abnormal condition through the monitoring device can go to the carpeted area at once to provide necessary assistance, but if a conventional motion sensing carpet is laid extensively in a house, or even in a large nursing home of several stories and with differently-sized partitioned areas on each floor, a caregiver spotting an abnormal condition through the monitoring device will have problem identifying the location of the abnormal condition, let alone reaching the site at the earliest possible time to deal with the situation. The problem can be solved to a certain degree by dividing the large area into a plurality of small ones, monitoring each small area with a separate monitoring device, and using a host device to collect the information gathered by each monitoring device. This solution, though feasible, will not work well if the entire area to be monitored is not sufficiently divided; however, if the entire area is overly divided, the cost of purchasing the monitoring devices will be considerable, which is by no means ideal.

In summary of the above, a conventional motion sensing carpet allows a caregiver to rapidly know the occurrence of an accident, but if the carpet is applied to an extensive area, the caregiver may find it difficult to locate the accident at once and hence cannot get to the site of the accident as soon as possible. It is important, therefore, for those in the industry to design a monitoring system of motion sensing carpets. It is desirable to define a topology matrix for the motion sensing carpets in use so that a caregiver not only can be alerted to the emergence of an accident promptly, but also can locate the accident without delay.

### BRIEF SUMMARY OF THE INVENTION

In view of and in order to overcome the aforementioned drawbacks of the conventional motion sensing carpets, the inventor of the invention incorporated years of practical experience in the industry into extensive research and experiment and finally succeeded in developing a monitoring system of motion sensing carpets as disclosed herein.

The present invention provides a monitoring system of motion sensing carpets. The monitoring system includes a plurality of motion sensing carpets and a monitoring device (e.g., a computer). Each of the motion sensing carpets is provided with a control module, a storage module, a sensor module, and a plurality of information transmission modules, wherein: the control module is separately electrically connected to the storage module, the sensor module, and the information transmission modules; the storage module stores an identification tag; the sensing module generates a sensing signal when the motion sensing carpet is subjected to pressure; the control module can read the identification tag, receive the sensing signal, and send the identification tag or the sensing signal to an adjacent motion sensing carpet through one of the information transmission modules; and the information transmission modules are provided at the periphery of the motion sensing carpet, correspond to different pieces of position information respectively, and, when the motion sensing carpet is joined with another motion sensing carpet, can connect with and transmit information to and from the corresponding information transmission module of that other motion sensing carpet. The monitoring device stores a queue and a topology matrix and is electrically connected to one of the motion sensing carpets such that the motion sensing carpet electrically connected with the monitoring device serves as a control unit while all the other motion sensing carpets, which are directly or indirectly joined to the control unit, serve as auxiliary units. The monitoring device carries out a topology algorithm whereby the monitoring device sequentially enters into the queue all the identification tags obtained by searching and establishes the topology matrix of the motion sensing carpets in a stepwise manner so as to obtain the relative location of each motion sensing carpet. When any of the motion sensing carpets is subjected to pressure and generates a sensing signal, the monitoring device can know from the topology matrix the actual location of the motion sensing carpet generating the sensing signal. Thus, by means of the topology algorithm, the relative location of each motion sensing carpet is obtained, and the monitoring device can know from the topology matrix the actual location of the motion sensing carpet which generates a sensing signal because of an applied pressure. For example, if a senior member or a child in the family topples over on one of the motion sensing carpets, the exact location of the accident can be rapidly known according to the sensing signal.

### BRIEF DESCRIPTION OF THE SEVERAL VIEWS OF THE DRAWINGS

The objectives as well as the technical features and effects of the present invention will be best understood by referring to the following detailed description of some illustrative embodiments in conjunction with the accompanying drawings, in which:

FIG. 1 schematically shows the connections between the major elements of the present invention;

FIG. 2 schematically shows how the motion sensing carpets of the present invention are joined to one another;

FIG. 3 is a flowchart showing the major steps of the present invention; and

FIG. 4 is a conceptual diagram showing how a queue and a topology matrix are established by the topology algorithm of the present invention.

### DETAILED DESCRIPTION OF THE INVENTION

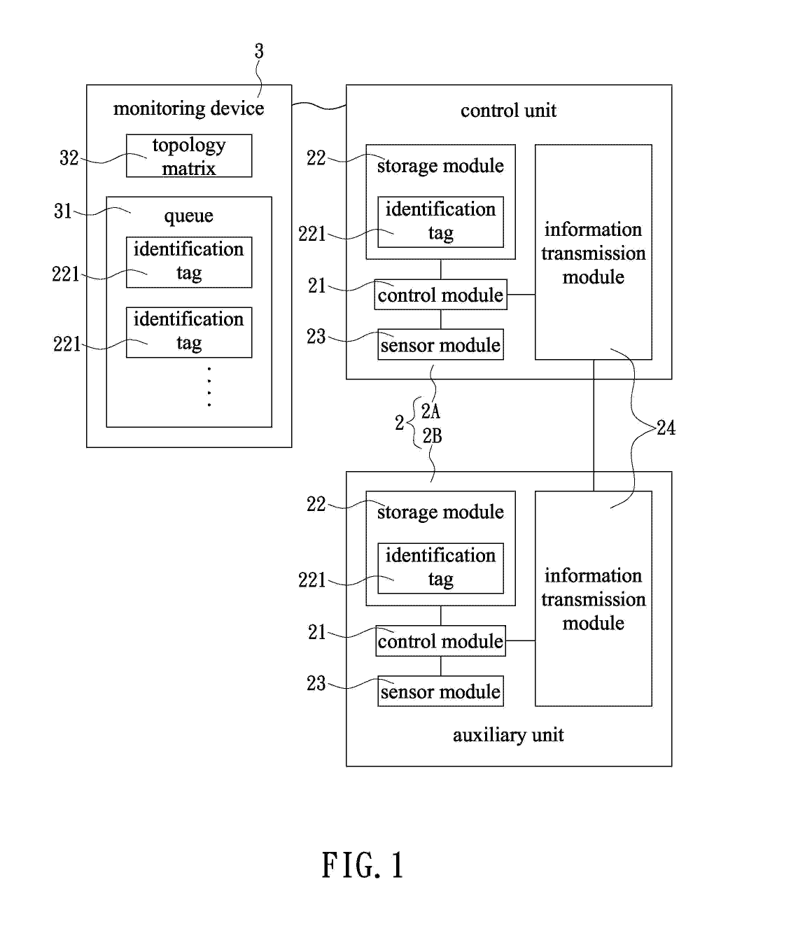

The present invention provides a monitoring system of motion sensing carpets. Referring to FIG. 1, the monitoring system includes a plurality of motion sensing carpets 2 and a monitoring device 3 (e.g., a computer) so that a caregiver can know the exact locations of the motion sensing carpets 2 through the monitoring device 3. The motion sensing carpets 2 are of the same specifications and are each provided with a control module 21, a storage module 22, a sensor module 23, and a plurality of information transmission modules 24. In each motion sensing carpet 2, the control module 21 is electrically connected to the storage module 22, the sensor module 23, and the information transmission modules 24 in order to transmit signals to and from the storage module 22, the sensor module 23, and the information transmission modules 24; the storage module 22 stores an identification tag 221 corresponding to the motion sensing carpet 2; the control module 21 can read the identification tag 221 from the storage module 22; and when the motion sensing carpet 2 is subjected to pressure, the sensor module 23 generates a sensing signal and sends the sensing signal to the control module 21.

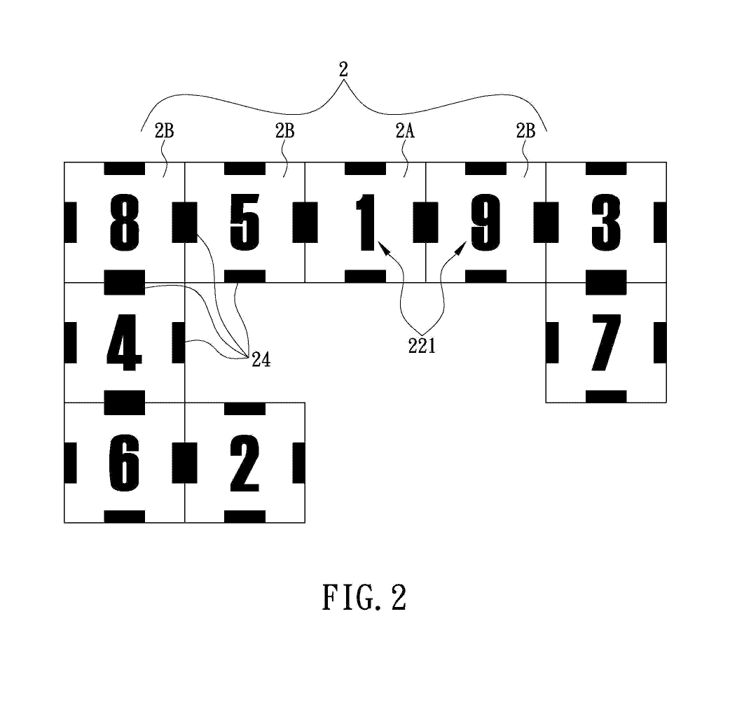

Referring to FIG. 1 and FIG. 2, the motion sensing carpets 2 are rectangular, and the information transmission modules 24 of each motion sensing carpet 2 are respectively provided at the four sides of the motion sensing carpet 2 and correspond respectively to different position information (e.g., upper side, lower side, left side, and right side). In this preferred embodiment, each information transmission module 24 includes a digital input pin and a digital output pin (not shown) so that, when two motion sensing carpets 2 are joined to each other, the digital input pin of one of the two motion sensing carpets 2 can be connected with the digital output pin of the other motion sensing carpet 2 in order for the one of the two motion sensing carpets 2 to know that it is joined with an adjacent motion sensing carpet 2. In practice, however, the configuration of the information transmission modules 24 is not limited to the foregoing; other equivalent configurations are also feasible to enable the information transmission modules 24 of each motion sensing carpet 2 to know if the motion sensing carpet 2 is joined with another motion sensing carpet 2. When two motion sensing carpets 2 are joined together, these two adjacent motion sensing carpets 2 can transmit information to and from each other through the connected information transmission modules 24. In other words, when the plural motion sensing carpets 2 are joined together and all the corresponding information transmission modules 24 are connected, the control module 21 of any motion sensing carpet 2 can send the identification tag 221 of the motion sensing carpet 2 or a received sensing signal through one of the information transmission modules 24 of the motion sensing carpet 2 to another motion sensing carpet 2 after reading the identification tag 221 or receiving the sensing signal.

Referring back to FIG. 1, the monitoring device 3 stores a queue 31 and a topology matrix 32 and is electrically connected to one of the motion sensing carpets 2. The motion sensing carpet 2 electrically connected with the monitoring device 3 functions as a control unit 2A. All the other motion sensing carpets 2, which are either directly or indirectly connected to the control unit 2A, serve as auxiliary units 2B. The monitoring device 3 of the present invention can drive the control module 21 of each motion sensing carpet 2 to detect the information transmission modules 24 of the motion sensing carpet 2. Then, based on the detection results, and by means of the breadth-first search (BFS) algorithm and the first-in first-out technique, the monitoring device 3 sequentially establishes the queue 31 corresponding to the motion sensing carpets 2. In the meantime, the monitoring device 3 also establishes, in a stepwise manner according to the queue 31, the topology matrix 32 composed of all the motion sensing carpets 2. Once the queue 31 and the topology matrix 32 are established, the monitoring device 3 can rapidly determine the location of any motion sensing carpet 2 that generates a sensing signal.

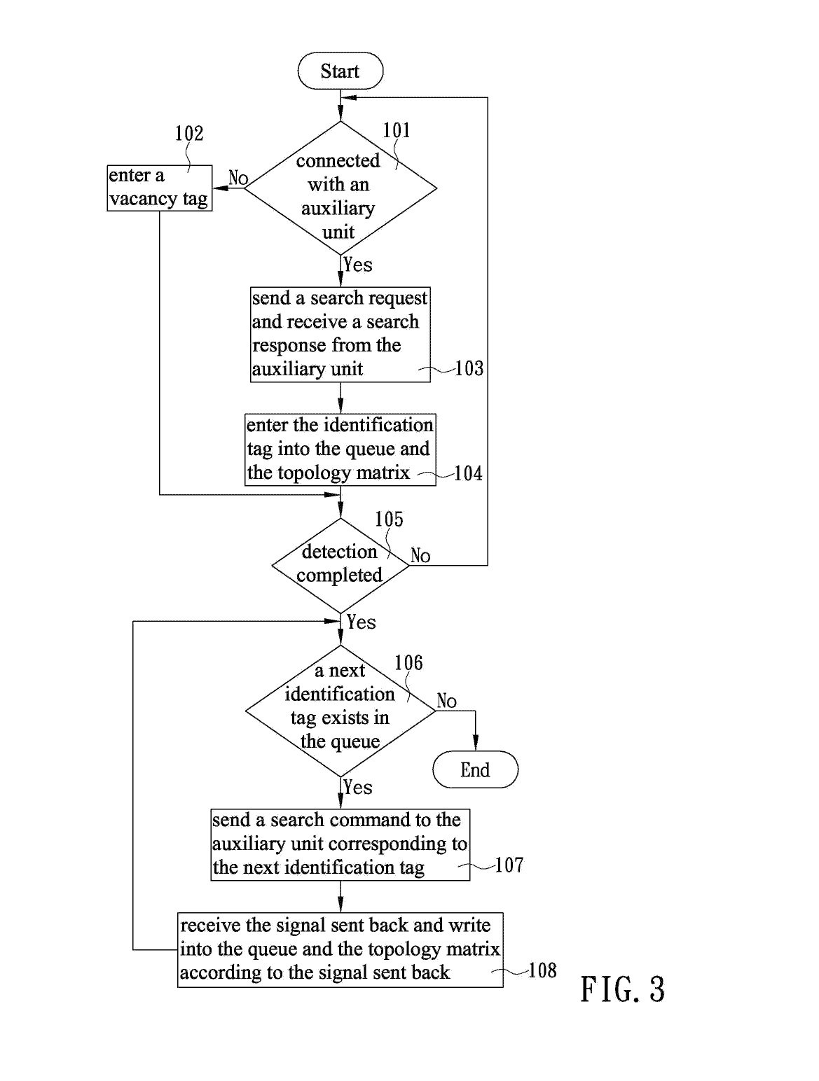

The process flow of the operation of the present invention is detailed below with reference to FIG. 3, which is a flowchart showing the major steps, in conjunction with the reference numerals in FIG. 1. According to the topology algorithm of the present invention, the monitoring device 3 performs the following steps:

(101) The monitoring device 3 drives the control module 21 of the control unit 2A to sequentially detect the information transmission modules 24 of the control unit 2A in order to determine whether any of the information transmission modules 24 is connected with one of the information transmission modules 24 of an adjacent auxiliary unit 2B. If no, step (102) is executed; if yes, step (103) is executed.

(102) According to the position information of the information transmission module 24 being detected, the monitoring device 3 enters a vacancy tag (e.g., the code 0) into the corresponding position in the topology matrix 32. Then, step (105) is executed.

(103) The monitoring device 3 sends a search request to the adjacent auxiliary unit 2B through the information transmission module 24 being detected, in order for the auxiliary unit 2B to send a search response to the monitoring device 3 according to the search request after receiving the search request. The search response includes the identification tag 221 corresponding to the auxiliary unit 2B and the position information of the information transmission module 24 receiving the search request. Then, step (104) is executed.

(104) After receiving the search response, the monitoring device 3 stores the identification tag 221 into the queue 31 in order and, based on the position information in the search response, enters the identification tag 221 into the corresponding positon in the topology matrix 32. Then, step (105) is executed.

(105) The monitoring device 3 determines whether all the information transmission modules 24 of the control unit 2A have been detected. If no, the process returns to step (101); if yes, step (106) is executed.

(106) The monitoring device 3 determines whether there is a next identification tag 221 in the queue 31. If yes, step (107) is executed; if no, the process ends.

(107) The monitoring device 3 reads the next identification tag 221 in the queue 31 and sends a search command to the auxiliary unit 2B corresponding to the identification tag 221, in order for this auxiliary unit 2B to sequentially detect its information transmission modules 24 according to the search command and either send to the monitoring device 3 a vacancy response including the position information of the information transmission module 24 being detected or send a search request to an adjacent auxiliary unit through the information transmission module 24 being detected and then relay a search response to the monitoring device 3. Upon completing the detection of all of its information transmission modules 24, this auxiliary unit 2B sends a completion response to the monitoring device 3 and enters a non-responsive state, in which the auxiliary unit 2B will not send any search response to the monitoring device 3 if a search request is subsequently received from another auxiliary unit 2B.

(108) Upon receiving the vacancy response, and according to the position information of the information transmission module 24 being detected in the vacancy response, the monitoring device 3 enters the vacancy tag into the corresponding position in the topology matrix 32. Or upon receiving the search response, the monitoring device 3 stores the identification tag 221 in the search response into the queue 31 in order and, based on the position information in the search response, enters the identification tag 221 into the corresponding position in the topology matrix 32. The process returns to step (106) if the monitoring device 3 receives the completion response.

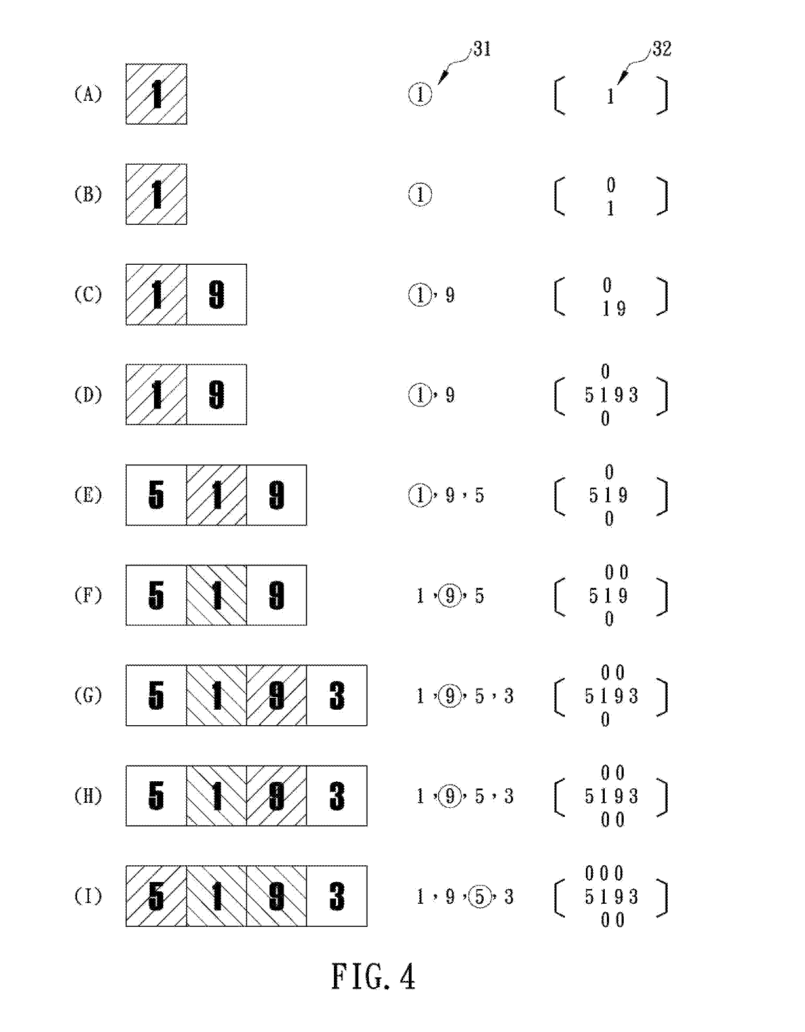

To enable more intuitive understanding of the topology algorithm of the present invention, or more particularly the actual process in which the queue 31 and the topology matrix 32 are established by the BFS algorithm and the first-in first-out technique, an example is given below with reference to the conceptual diagram of FIG. 4, the reference numerals in FIG. 1, and the relationship between the joined motion sensing carpets 2 in FIG. 2, so as to shed light on the steps of establishing the queue 31 and the topology matrix 32. In FIG. 4, the motion sensing carpets 2 in the left column that are marked with “upper right-to-lower left” hatching lines and the motion sensing carpets 2 corresponding to the circled identification tags 221 in the middle column are detecting their respective information transmission modules 24. On the other hand, the motion sensing carpets 2 in the left column of FIG. 4 that are marked with “upper left-to-lower right” hatching lines are in the non-responsive state. To begin with, the monitoring device 3 stores the identification tag 221 corresponding to the control unit 2A (in this example, the motion sensing carpet 2 with the identification tag 221 of No.1) into the queue 31 and also enters the identification tag 221 of the control unit 2A into the corresponding position in the topology matrix 32. Then, the monitoring device 3 drives the control module 21 of the control unit 2A to detect the auxiliary units 2B joined to the control unit 2A, starting from the upper side of the control unit 2A to the right side, the lower side, and left side, in that order. If no auxiliary unit 2B is detected at a certain position, the monitoring device 3 enters the code 0 into the corresponding position in the topology matrix 32. If an auxiliary unit 2B is detected at a certain position, the monitoring device 3 enters the identification tag 221 corresponding to the auxiliary unit 2B into the queue 31 and also into the corresponding position in the topology matrix 32.

Once the control unit 2A has detected all of its information transmission modules 24, the monitoring device 3 reads the next identification tag 221 in the queue 31 and drives the auxiliary unit 2B corresponding to this identification tag 221 (in this example, the motion sensing carpet 2 with the identification tag 221 of No. 9), in order for the control module 21 of this auxiliary unit 2B to detect the auxiliary units 2B joined to this auxiliary unit 2B, starting from the upper side of this auxiliary unit 2B to the right side, the lower side, and left side, in that order. After this auxiliary unit 2B has detected all of its information transmission modules 24, the monitoring device 3 sequentially reads the following identification tags 221 in the queue 31 (in this example, the identification tags 221 of Nos. 5, 3, 8, 7, 4, 6, and 2, in that order) in order to sequentially drive the corresponding auxiliary units 2B to detect their respective information transmission modules 24. The foregoing process stops after the auxiliary units 2B corresponding to all the identification tags 221 in the queue 31 have finished detecting their respective information transmission modules 24.

Through the topology algorithm described above, the relative locations of all the motion sensing carpets 2 can be obtained, and the monitoring device 3 can know from the topology matrix 32 the actual location of any motion sensing carpet 2 that is subjected to pressure and hence generates a sensing signal. Thus, if an elderly family member or child accidently falls on any of the motion sensing carpets 2, the location of the accident can be rapidly known according to the sensing signal.

While the invention herein disclosed has been described by means of specific embodiments, numerous modifications and variations could be made thereto by those skilled in the art without departing from the scope of the invention set forth in the claims.

What is claimed is:

1. A monitoring system of motion sensing carpets, comprising:
a plurality of motion sensing carpets joined to one another, each said motion sensing carpet being provided with a control module, a storage module, a sensor module, and a plurality of information transmission modules, wherein the control module is separately electrically connected to the storage module, the sensor module, and the information transmission modules; the storage module stores an identification tag; the sensor module generates a sensing signal when the motion sensing carpet is subjected to pressure; the control module is configured for reading the identification tag, receiving the sensing signal, and sending the identification tag or the sensing signal to an adjacent said motion sensing carpet through one of the information transmission modules; and the information transmission modules are provided at a periphery of the motion sensing carpet, each correspond to a piece of position information, and are respectively connected to corresponding ones of the information transmission modules of all said motion sensing carpets joined to the motion sensing carpet so as to enable transmission of information between the motion sensing carpet and each said motion sensing carpet joined to the motion sensing carpet; and
a monitoring device storing a queue and a topology matrix and electrically connected to one of the motion sensing carpets such that the motion sensing carpet electrically connected with the monitoring device serves as a control unit while all the other motion sensing carpets, either directly or indirectly joined to the control unit, serve as auxiliary units, wherein, according to a topology algorithm, the monitoring device sequentially enters into the queue said identification tags obtained by searching and establishes the topology matrix of the motion sensing carpets in a stepwise manner so as to obtain relative locations of the motion sensing carpets and, when any of the motion sensing carpets is subjected to pressure and hence generates a said sensing signal, to know from the topology matrix an actual location of the motion sensing carpet generating the sensing signal.

2. The monitoring system of claim 1, wherein the motion sensing carpets are rectangular, and each said information transmission module is provided at one of the four sides of a said motion sensing carpet.

3. The monitoring system of claim 1, wherein each said information transmission module comprises a digital input pin and a digital output pin so that whether a said motion sensing carpet is joined with an adjacent said motion sensing carpet can be determined via the digital input pin and the digital output pin of the motion sensing carpet.

4. The monitoring system of claim 2, wherein each said information transmission module comprises a digital input pin and a digital output pin so that whether a said motion sensing carpet is joined with an adjacent said motion sensing carpet can be determined via the digital input pin and the digital output pin of the motion sensing carpet.

5. The monitoring system of claim 3, wherein, according to the topology algorithm, the monitoring device performs the steps of:
driving the control module of the control unit to sequentially detect the information transmission modules of the control unit in order to determine whether any of the information transmission modules of the control unit is connected with a corresponding one of the information transmission modules of an adjacent said auxiliary unit;
entering a vacancy tag into a position in the topology matrix that corresponds to the position information of the information transmission module being detected, if the information transmission module being detected is not connected with any said information transmission module of the adjacent auxiliary unit;
sending a search request to the adjacent auxiliary unit through the information transmission module being detected, if the information transmission module being detected is connected with the corresponding one of the information transmission modules of the adjacent auxiliary unit, in order for the adjacent auxiliary unit to send a search response to the monitoring device upon receipt of and according to the search request, wherein the search response includes the identification tag corresponding to the adjacent auxiliary unit and the position information corresponding to the information transmission module receiving the search request; and
storing the identification tag in the search response into the queue in order, and entering the identification tag in the search response into a position in the topology matrix that corresponds to the position information in the search response, upon receipt of the search response.

6. The monitoring system of claim 4, wherein, according to the topology algorithm, the monitoring device performs the steps of:
driving the control module of the control unit to sequentially detect the information transmission modules of the control unit in order to determine whether any of the information transmission modules of the control unit is connected with a corresponding one of the information transmission modules of an adjacent said auxiliary unit;
entering a vacancy tag into a position in the topology matrix that corresponds to the position information of the information transmission module being detected, if the information transmission module being detected is not connected with any said information transmission module of the adjacent auxiliary unit;
sending a search request to the adjacent auxiliary unit through the information transmission module being detected, if the information transmission module being detected is connected with the corresponding one of the information transmission modules of the adjacent auxiliary unit, in order for the adjacent auxiliary unit to send a search response to the monitoring device upon receipt of and according to the search request, wherein the search response includes the identification tag corresponding to the adjacent auxiliary unit and the position information corresponding to the information transmission module receiving the search request; and
storing the identification tag in the search response into the queue in order, and entering the identification tag in the search response into a position in the topology matrix that corresponds to the position information in the search response, upon receipt of the search response.

7. The monitoring system of claim 5, wherein, according to the topology algorithm, the monitoring device further performs the steps of:
driving the control module of the control unit to detect another said information transmission module of the control unit;
reading a next said identification tag in the queue, and driving the auxiliary unit corresponding to the next identification tag through the control unit, if all the information transmission modules of the control unit have been detected; and
ending the topology algorithm if the next identification tag does not exist.

8. The monitoring system of claim 6, wherein, according to the topology algorithm, the monitoring device further performs the steps of:
driving the control module of the control unit to detect another said information transmission module of the control unit;
reading a next said identification tag in the queue, and driving the auxiliary unit corresponding to the next identification tag through the control unit, if all the information transmission modules of the control unit have been detected; and
ending the topology algorithm if the next identification tag does not exist.

9. The monitoring system of claim 7, wherein, according to the topology algorithm, the monitoring device further performs the step of:
sending a search command to the auxiliary unit corresponding to the next identification tag, in order for the auxiliary unit corresponding to the next identification tag to sequentially detect the information transmission modules of the auxiliary unit corresponding to the next identification tag according to the search command and either send to the monitoring device a vacancy response including the position information of the information transmission module being detected or send a said search request to an adjacent said auxiliary unit through the information transmission module being detected and subsequently relay a said search response to the monitoring device.

10. The monitoring system of claim 8, wherein, according to the topology algorithm, the monitoring device further performs the step of:
sending a search command to the auxiliary unit corresponding to the next identification tag, in order for the auxiliary unit corresponding to the next identification tag to sequentially detect the information transmission modules of the auxiliary unit corresponding to the next identification tag according to the search command and either send to the monitoring device a vacancy response including the position information of the information transmission module being detected or send a said search request to an adjacent said auxiliary unit through the information transmission module being detected and subsequently relay a said search response to the monitoring device.

11. The monitoring system of claim 9, wherein after all the information transmission modules of the auxiliary unit corresponding to the next identification tag have been detected, the auxiliary unit corresponding to the next identification tag sends a completion response to the monitoring device and enters a non-responsive state, in which the auxiliary unit corresponding to the next identification tag will not send any said search response to the monitoring device upon subsequent receipt of a said search request sent by another said auxiliary unit.

12. The monitoring system of claim 10, wherein after all the information transmission modules of the auxiliary unit corresponding to the next identification tag have been detected, the auxiliary unit corresponding to the next identification tag sends a completion response to the monitoring device and enters a non-responsive state, in which the auxiliary unit corresponding to the next identification tag will not send any said search response to the monitoring device upon subsequent receipt of a said search request sent by another said auxiliary unit.

13. The monitoring system of claim 11, wherein, according to the topology algorithm, the monitoring device further performs the step of:
entering the vacancy tag into a position in the topology matrix that corresponds to the position information of the information transmission module being detected in the vacancy response, upon receipt of the vacancy response;
storing the identification tag in the search response into the queue in order, and entering the identification tag in the search response into a position in the topology matrix that corresponds to the position information in the search response, upon receipt of the search response;
reading a following said identification tag in the queue, and driving the auxiliary unit corresponding to the following identification tag through the control unit, upon receipt of the completion response; or
ending the topology algorithm if the following identification tag does not exist.

14. The monitoring system of claim 12, wherein, according to the topology algorithm, the monitoring device further performs the step of:
entering the vacancy tag into a position in the topology matrix that corresponds to the position information of the information transmission module being detected in the vacancy response, upon receipt of the vacancy response;
storing the identification tag in the search response into the queue in order, and entering the identification tag in the search response into a position in the topology matrix that corresponds to the position information in the search response, upon receipt of the search response;
reading a following said identification tag in the queue, and driving the auxiliary unit corresponding to the following identification tag through the control unit, upon receipt of the completion response; or
ending the topology algorithm if the following identification tag does not exist.
